(19) United States
(12) Patent Application Publication
(10) Pub. No.: US 2018/0037942 A1
(43) Pub. Date: Feb. 8, 2018

(54) ENZYME-INDEPENDENT MOLECULAR INDEXING

(71) Applicant: Cellular Research, Inc., Menlo Park, CA (US)

(72) Inventor: Glenn Fu, Menlo Park, CA (US)

(21) Appl. No.: 15/665,906

(22) Filed: Aug. 1, 2017

Related U.S. Application Data
(60) Provisional application No. 62/370,620, filed on Aug. 3, 2016.

(51) Int. Cl. C12Q 1/68

(52) U.S. Cl. CPC C12Q 1/6837; C12Q 1/6874

(57) ABSTRACT

The present disclosure relates to compositions, methods and kits for quantitative analysis of a plurality of nucleic acid target molecules in a sample. In some embodiments, the methods comprise associating molecular label sequences with target nucleic acid molecules without an enzymatic reaction.

### RELATED APPLICATIONS

The present application claims priority under 35 U.S.C. §119(e) to U.S. Provisional Application No. 62/370,620, filed on Aug. 3, 2016. The content of this related application is herein expressly incorporated by reference in its entirety.

### BACKGROUND

Molecular label sequences, or molecular barcodes, have been used to digitally count nucleic acid target molecules, such as mRNAs, in a sample. In order to associate the molecular label sequences with the nucleic acid targets, an enzymatic reaction, such as reverse transcription or ligation, has been used. However, the efficiency of the enzymatic reactions can be influenced by a number of factors, such as the quality of the nucleic acid targets in a sample, the presence of inhibitory ions or impurities, etc. Therefore, there is a need for an enzyme-independent method to associating molecular label sequences with target nucleic acid molecules for quantitative analysis of target nucleic acid molecules.

### SUMMARY

Some embodiments disclosed herein provide methods of quantitative analysis of a plurality of nucleic acid target molecules in a sample comprising: providing a sample comprising a plurality of nucleic acid target molecules; providing a plurality of oligonucleotide probes, wherein each of the plurality of oligonucleotide probes comprises a target specific region, a molecular label sequence, and a binding site for a first universal primer, wherein the molecular label sequence is selected from a diverse set of unique molecular label sequences; contacting the plurality of oligonucleotide probes with the plurality of nucleic acid target molecules for hybridization; removing oligonucleotide probes that are not hybridized to the plurality of nucleic acid target molecules; amplifying oligonucleotide probes that are hybridized to the plurality of nucleic acid target molecules using the first universal primer to generate a plurality of amplicons, wherein each of the plurality of amplicons comprises a target specific region and a molecular label sequence; and determining the number of unique molecular label sequences for each target specific region, whereby the quantity of each nucleic acid target molecule in the sample is determined. The the molecular label sequences of two of the plurality of oligonucleotide probes can be different.

In some embodiments, the methods further comprise immobilizing the plurality of nucleic acid target molecules on a solid support via an affinity moiety and a binding partner of the affinity moiety. In some embodiments, the plurality of nucleic acid target molecules comprises the affinity moiety. In some embodiments, the affinity moiety is a functional group of biotin, streptavidin, heparin, an aptamer, a click-chemistry moiety, digoxigenin, primary amine, carboxyl, hydroxyl, aldehyde, ketone, or any combination thereof. In some embodiments, the plurality of nucleic acid target molecules is biotinylated. In some embodiments, the plurality of nucleic acid target molecules is hybridized to a plurality of biotinylated capture probes. In some embodiments, each of the plurality of biotinylated capture probes comprises a second target specific region. In some embodiments, the second target specific region comprises poly dT. In some embodiments, the solid support comprises the binding partner of the affinity moiety. In some embodiments, removing oligonucleotide probes that are not hybridized to the plurality of nucleic acid target molecules comprises washing the solid support. In some embodiments, each of the plurality of oligonucleotide probes comprises a binding site for a second universal primer. In some embodiments, the amplifying comprises PCR amplification of at least a portion of the oligonucleotide probes that are hybridized to the plurality of nucleic acid target molecules using the first universal primer and the second universal primer. In some embodiments, each of the plurality of oligonucleotide probes comprises a cellular label, a sample label, a location label, or any combination thereof. In some embodiments, the target specific region comprises 20 nt to 500 nt. In some embodiments, the sample comprises a single cell, a plurality of cells, a tissue sample, or any combination thereof. In some embodiments, the diverse set of unique molecular label sequences comprises at least 100 unique molecular label sequences. In some embodiments, the diverse set of unique molecular label sequences comprises at least 1,000 unique molecular label sequences. In some embodiments, the diverse set of unique molecular label sequences comprises at least 10,000 unique molecular label sequences. In some embodiments, at least 10 of the plurality of oligonucleotide probes comprise different target specific regions. In some embodiments, at least 100 of the plurality of oligonucleotide probes comprise different target specific regions. In some embodiments, at least 1,000 of the plurality of oligonucleotide probes comprise different target specific regions. In some embodiments, each of the plurality of RNA target molecules hybridizes to a single target specific region. In some embodiments, each of the plurality of RNA target molecules hybridizes to more than one different target specific regions. In some embodiments, the methods further comprise sequencing the plurality of amplicons. In some embodiments, the sequencing comprises sequencing at least a portion of the molecular label sequence and at least a portion of the target specific region. In some embodiments, the methods further comprise associating the sequence of the molecular label sequence with the sequence of the target specific region.

Some embodiments disclosed herein provide kits for quantitative analysis of a plurality of nucleic acid target molecules in a sample comprising a plurality of oligonucleotide probes, wherein each of the plurality of oligonucleotide probes comprises a target specific region, a molecular label sequence, a binding site for a first universal primer, and a binding site for a second universal primer, wherein the molecular label sequence is selected from a diverse set of unique molecular label sequences. The molecular label sequences of two of the plurality of oligonucleotide probes can be different.

In some embodiments, the kits further comprise a plurality of capture probes each comprising a second target specific region. In some embodiments, the plurality of capture probes is biotinylated. In some embodiments, the second target specific region comprises poly dT. In some embodiments, each of the plurality of oligonucleotide probes comprises a cellular label, a sample label, a location label, or any combination thereof. In some embodiments, the target specific region comprises 20 nt to 500 nt. In some embodiments, the diverse set of unique molecular label sequences comprises at least 100 unique molecular label sequences. In some embodiments, the diverse set of unique molecular label sequences comprises at least 1,000 unique molecular label sequences. In some embodiments, the diverse set of unique molecular label sequences comprises at least 10,000 unique molecular label sequences. In some embodiments, at least 10 of the plurality of oligonucleotide probes comprise different target specific regions. In some embodiments, at least 100 of the plurality of oligonucleotide probes comprise different target specific regions. In some embodiments, at least 1,000 of the plurality of oligonucleotide probes comprise different target specific regions. In some embodiments, each of the plurality of oligonucleotide probes comprises a different molecular label sequence-target specific region combination. In some embodiments, the kits comprise at least 1,000 oligonucleotide probes. In some embodiments, the kits comprise at least 10,000 oligonucleotide probes. In some embodiments, the kits comprise at least 100,000 oligonucleotide probes. In some embodiments, the kits comprise at least 1,000,000 oligonucleotide probes.

### BRIEF DESCRIPTION OF THE DRAWINGS

FIG. 1 shows a schematic illustration of an exemplary method of labeling a target nucleic acid with a molecular barcode.

### DETAILED DESCRIPTION

### Definitions

Unless otherwise defined, all technical terms used herein have the same meaning as commonly understood by one of ordinary skill in the art in the field to which this disclosure belongs. As used in this specification and the appended claims, the singular forms “a,” “an,” and “the” include plural references unless the context clearly dictates otherwise. Any reference to “or” herein is intended to encompass “and/or” unless otherwise stated.

As used herein the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “attached”, or “immobilized” to one another or to a common solid or semisolid surface. An association may refer to covalent or non-covalent means for attaching labels to solid or semi-solid supports such as beads. An association may comprise hybridization between a target and a label.

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, the terms “complement”, “complementary”, and “reverse complement” can be used interchangeably. It is understood from the disclosure that if a molecule can hybridize to another molecule it may be the complement of the molecule that is hybridizing.

As used herein, the term “digital counting” can refer to a method for estimating a number of target molecules in a sample. Digital counting can include the step of determining a number of unique labels that have been associated with targets in a sample. This stochastic methodology transforms the problem of counting molecules from one of locating and identifying identical molecules to a series of yes/no digital questions regarding detection of a set of predefined labels.

As used herein, the term “label” or “labels” can refer to nucleic acid codes associated with a target within a sample. A label can be, for example, a nucleic acid label. A label can be an entirely or partially amplifiable label. A label can be entirely or partially sequenceable label. A label can be a portion of a native nucleic acid that is identifiable as distinct. A label can be a known sequence. A label can comprise a junction of nucleic acid sequences, for example a junction of a native and non-native sequence. As used herein, the term “label” can be used interchangeably with the terms, “index”, “tag,” or “label-tag.” Labels can convey information. For example, in various embodiments, labels can be used to determine an identity of a sample, a source of a sample, an identity of a cell, and/or a target.

As used herein, a “nucleic acid” can generally refer to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g. altered backgone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or flurescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone of the nucleic acid can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (i.e. morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C=C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,′:4,5)pyrrolo[2,3-d]pyrimidin-2-one).

As used herein, the term “sample” can refer to a composition comprising targets. Suitable samples for analysis by the disclosed methods, devices, and systems include cells, single cells, tissues, organs, or organisms.

As used herein, the term “sampling device” or “device” can refer to a device which may take a section of a sample and/or place the section on a substrate. A sample device can refer to, for example, a fluorescence activated cell sorting (FACS) machine, a cell sorter machine, a biopsy needle, a biopsy device, a tissue sectioning device, a microfluidic device, a blade grid, and/or a microtome.

As used herein, the term “solid support” can refer to discrete solid or semi-solid surfaces to which a plurality of stochastic barcodes may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A plurality of solid supports spaced in an array may not comprise a substrate. A solid support may be used interchangeably with the term “bead.” As used herein, “solid support” and “substrate” can be used interchangeably.

As used here, the term “target” can refer to a composition which can be associated with a stochastic barcode. Exemplary suitable targets for analysis by the disclosed methods, devices, and systems include oligonucleotides, DNA, RNA, mRNA, microRNA, tRNA, and the like. Targets can be single or double stranded. In some embodiments, targets can be or comprise proteins. In some embodiments, targets are or comprise lipids. As used herein, “target” can be used interchangeably with “species”.

### Methods of Quantitative Analysis of Nucleic Acid Target Molecules

This disclosure provides methods that allow for associating molecular label sequences with, and quantitative analysis of, nucleic acid target molecules, such as mRNA molecules. In some embodiments, the nucleic acid target molecules can be associated with molecular label sequences without using an enzyme, such as a polymerase, a ligase, a reverse transcriptase, etc. In some embodiments, the nucleic acid target molecules can be associated with molecular label sequences by contacting a plurality of oligonucleotide probes with a plurality of nucleic acid target molecules for hybridization, and removing oligonucleotide probes that are not hybridized to the plurality of nucleic acid target molecules. In some embodiments, the oligonucleotide probes that are hybridized to the plurality of nucleic acid target molecules are amplified to generate a plurality of amplicons, so that the molecular label sequences are associated with the nucleic acid target molecules.

In some embodiments, removing oligonucleotide probes that are not hybridized to the plurality of nucleic acid target molecules comprises immobilizing the plurality of nucleic acid target molecules on a solid support, such as beads. For example, the plurality of nucleic acid target molecules can be immobilized on the solid support via an affinity moiety and its binding partner. In some embodiments, the affinity moiety or its binding partner is a functional group selected from the group consisting of biotin, streptavidin, heparin, an aptamer, a click-chemistry moiety, digoxigenin, primary amine(s), carboxyl(s), hydroxyl(s), aldehyde(s), ketone(s), and any combination thereof. The affinity moiety can be directly conjugated to the target nucleic acid molecules, for example, by biotinylation. In some embodiments, the affinity moiety can be conjugated to a capture probe comprising a second target specific region. The second target specific region can bind to the same target nucleic acid molecules that the oligonucleotide probes bind to. In some embodiments, the second target specific region can bind to all target nucleic acid molecules in a sample. For example, the second target specific region can comprise an oligo dT which can hybridize with mRNAs comprising poly-adenylated ends. The second target specific region can be gene-specific. For example, the second target specific region can be configured to hybridize to a specific region of a target. The second target specific region can be, or be at least, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 or more nucleotides in length. The second target specific region can be at most 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 or more nucleotides in length. In some embodiments, the second target specific region can be from 5-30 nucleotides in length. In some embodiments, the conjugated target nucleic acid molecule and/or the conjugated capture probe can be immobilized on a solid support coated with the binding partner of the affinity moiety, for example, streptavidin-coated beads. In some embodiments, the oligonucleotide probes that are not hybridized to the plurality of nucleic acid target molecules can be removed by washing the solid support.

In some embodiments, the hybridized oligonucleotide probes can be used as a template for amplification. One or more nucleic acid amplification reactions may be performed to create multiple copies of the molecular labeled target nucleic acid molecules. In some embodiments, the amplification can be performed using one or more universal primers that bind to one or more binding sites on the oligonucleotide probe.

Amplification may be performed in a multiplexed manner, wherein multiple nucleic acid sequences are amplified simultaneously. The amplification reactions may comprise amplifying at least a portion of the molecular label sequence and at least a portion of the target specific region. The amplification reactions may comprise amplifying at least a portion of a sample label, a cellular label, a spatial label, or a combination thereof. The amplification reactions may comprise amplifying at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the hybridized oligonucleotide probes.

In some embodiments, amplification can be performed using a polymerase chain reaction (PCR). As used herein, PCR may refer to a reaction for the in vitro amplification of specific DNA sequences by the simultaneous primer extension of complementary strands of DNA. As used herein, PCR may encompass derivative forms of the reaction, including but not limited to, RT-PCR, real-time PCR, nested PCR, quantitative PCR, multiplexed PCR, digital PCR, and assembly PCR.

Amplification of the labeled nucleic acids can comprise non-PCR based methods. Examples of non-PCR based methods include, but are not limited to, multiple displacement amplification (MDA), transcription-mediated amplification (TMA), whole transcriptome amplification (WTA), whole genome amplification (WGA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), and a Qβ replicase (Qβ) method, use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and ramification extension amplification (RAM). In some instances, the amplification may not produce circularized transcripts.

Amplification may comprise use of one or more non-natural nucleotides. Non-natural nucleotides may comprise photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). Non-natural nucleotides may be added to one or more cycles of an amplification reaction. The addition of the non-natural nucleotides may be used to identify products as specific cycles or time points in the amplification reaction.

The end products of the methods disclosed herein, such as a plurality of amplicons, are suitable for, for example, sequence identification, transcript counting, alternative splicing analysis, mutation screening, etc., in a high throughput manner. The methods disclosed herein can be used for associating a molecular label sequence with a plurality of target nucleic acids, e.g., a DNA molecule, an RNA molecule, an mRNA molecule or a cDNA molecule. In some embodiments, the target nucleic acids can be of low quality, such as being fragmented, contaminated with impurities, or of low quantity, such being from a sample of less than 1 ng, less than 100 pg, or less than 10 pg of nucleic acid molecules. For example, the plurality of target nucleic acids can comprise at least 1, at least 2, at least 3, at least 4, at least 5, at least 10, at least 100, at least 1,000, at least 10,000, at least 100,0000, at least 1,000,000, or more target nucleic acid molecules. In some embodiments, at least 10%, at least 20%, at least 30%, at least 40%, at least 50%, at least 60%, at least 70%, at least 80%, at least 90%, at least 95%, at least 98%, at least 99%, of the plurality of nucleic acid targets are associated with a molecular label sequence.

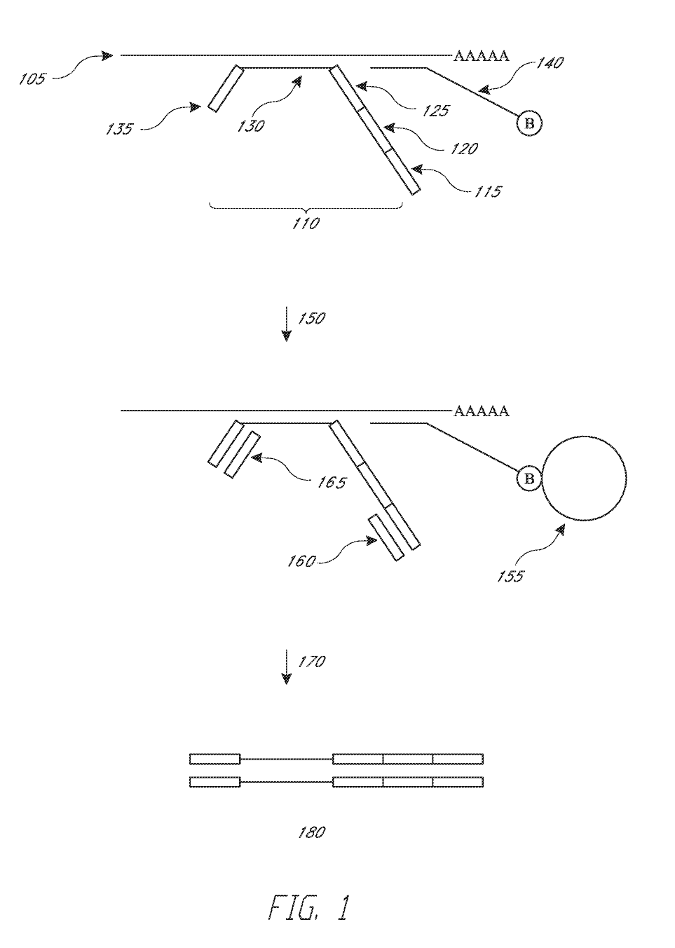

An exemplary method for labeling a target nucleic acid is illustrated in FIG. 1. As shown, an mRNA molecule 105 can be hybridized to an oligonucleotide 110 that can comprise a binding site 115 for a first universal primer, optionally a sample label 120, a molecular label 125, a target specific region 130, and a binding site 135 for a second universal primer. In some embodiments, a capture probe 140, which can be biotinylated, is also hybridized to the mRNA molecule 105. For example, the capture probe 140 can include a poly dT region which can hybridize to the poly A region of the mRNA molecule 105. As another example, the capture probe 140 can hybridize to the non-poly A region of the mRNA molecule 105. Unhybridized oligonucleotides 115 can be removed at 150 by using a streptavidin coated bead 155, which the biotinylated capture probe 140 can bind to. The hybridized oligonucleotide 110 can be amplified at 170 using a first universal primer 160 and a second universal primer 165 to produce a plurality of amplicons 180. Some or all of the plurality of amplicons 180 can be sequenced. The quantity of the mRNA molecule 105 can be determined, for example, based on the number of molecule labels 125 with different sequences associated with the sequence of the target specific region 120.

### Oligonucleotide Probes

The present disclosure provides a plurality of oligonucleotide probes for associating molecular label sequences with nucleic acid target molecules in a sample. The oligonucleotide probes disclosed herein can comprise one or more of a molecular label sequence, a target specific region, and a binding site for a universal primer. Without being bound by theory, the plurality of oligonucleotides comprises a unique set of molecular label sequence-target specific region combination. For example, each of the plurality of oligonucleotide probes may comprise a different molecular label sequence-target specific region combination. In some embodiments, the plurality of oligonucleotide probes comprises at least 1,000 oligonucleotide probes. In some embodiments, the plurality of oligonucleotide probes comprises at least 10,000 oligonucleotide probes. In some embodiments, the plurality of oligonucleotide probes comprises at least 100,000 oligonucleotide probes. In some embodiments, the plurality of oligonucleotide probes comprises at least 1,000,000 oligonucleotide probes. The oligonucleotide probes can have a variety of lengths. For example, an oligonucleotide probe can have a length that is, is about, is less than, is more than, 30 nt, 40 nt, 50 nt, 60 nt, 70 nt, 80 nt, 90 nt, 100 nt, 200 nt, 300 nt, 400 nt, 500 nt, 600 nt, 700 nt, 800 nt, 900 nt, 1,000 nt, or a range between any two of the above values.

An oligonucleotide probe can comprise one or more labels. Exemplary labels include, but are not limited to, a binding site for a universal primer, a cellular label, a molecular label, a sample label, a plate label, a spatial label, and/or a pre-spatial label. A molecular label sequence can comprise a 5′ amine that may link the molecular label sequence to a solid support. The oligonucleotide probe can comprise one or more of a binding site for a universal primer, a cellular label, and a molecular label. The binding site for a universal primer may be 5′-most label. The binding site for a universal primer may be the 3′-most label. In some embodiments, the oligonucleotide probe can comprise two binding sites for universal primers, which may be identical or different. In some instances, the binding site for a universal primer, the cellular label, and the molecular label are in any order. The oligonucleotide probe can comprise a target specific region. The target specific region can interact with a target (e.g., target nucleic acid, RNA, mRNA, DNA) in a sample. In some instances, the labels of the oligonucleotide probe (e.g., binding site for universal primer, dimension label, spatial label, cellular label, and molecular label sequence) may be separated by 1, 2, 3, 4, 5, 6, 7, 8, 9, 10, 11, 12, 13, 14, 15, 16, 17, 18, 19, or 20 or more nucleotides.

Each of the plurality of oligonucleotide probes may comprise a target specific region. In some embodiments, the target specific regions may comprise a nucleic acid sequence that hybridizes specifically to a target (e.g., target nucleic acid, target molecule, e.g., a cellular nucleic acid to be analyzed), for example to a specific gene sequence. In some embodiments, a target binding region may comprise a nucleic acid sequence that may attach (e.g., hybridize) to a specific location of a specific target nucleic acid. The target specific regions can have a variety of lengths. For example, an target specific region can have a length that is, is about, is less than, is more than, 5 nt, 6 nt, 7 nt, 8 nt, 9 nt, 10 nt, 20 nt, 30 nt, 40 nt, 50 nt, 60 nt, 70 nt, 80 nt, 90 nt, 100 nt, 200 nt, 300 nt, 400 nt, 500 nt, or a range between any two of the above values.

A target specific region can hybridize with a target nucleic acid molecule of interest. A target specific region can be gene-specific. For example, a target specific region can be configured to hybridize to a specific region of a target gene. In some embodiments, a target specific region can be specific for a variant of a gene, such as a mutation, a splice variant, an SNP site, etc. In some embodiments, a target specific region can be specific for a polymorphic location. In some embodiments, a target specific region can be specific for an allele.

An oligonucleotide probe may comprise a molecular label sequence. A molecular label sequence may comprise a nucleic acid sequence that provides identifying information for the specific type of target nucleic acid species hybridized to the oligonucleotide probe. A molecular label sequence may comprise a nucleic acid sequence that provides a counter for the specific occurrence of the target nucleic acid species hybridized to the oligonucleotide probe (e.g., target-binding region). In some embodiments, there may be as many as 10⁶ or more unique molecular label sequences in the plurality of oligonucleotide probes. In some embodiments, there may be as many as 10⁵ or more unique molecular label sequences in the plurality of oligonucleotide probes. In some embodiments, there may be as many as 10⁴ or more unique molecular label sequences in the plurality of oligonucleotide probes. In some embodiments, there may be as many as 10³ or more unique molecular label sequences in the plurality of oligonucleotide probes. In some embodiments, there may be as many as 10² or more unique molecular label sequences in the plurality of oligonucleotide probes. A molecular label sequence may be at least about 1, 2, 3, 4, 5, 10, 15, 20, 25, 30, 35, 40, 45, 50, or more nucleotides in length. A molecular label sequence may be at most about 300, 200, 100, 90, 80, 70, 60, 50, 40, 30, 20, 15, 12, 10, 9, 8, 7, 6, 5, 4, or fewer nucleotides in length.

An oligonucleotide probe can, in some embodiments, comprise one or more binding sites for universal primers. The one or more binding sites for universal primers may be the same for all the oligonucleotide probes in a plurality of oligonucleotide probes. In some embodiments, a binding site for a universal primer may comprise a nucleic acid sequence that is capable of hybridizing to a sequencing primer. Sequencing primers (e.g., universal sequencing primers) may comprise sequencing primers associated with high-throughput sequencing platforms. In some embodiments, a binding site for a universal primer may comprise a nucleic acid sequence that is capable of hybridizing to a PCR primer. In some embodiments, the binding site for a universal primer may comprise a nucleic acid sequence that is capable of hybridizing to a sequencing primer and a PCR primer. The nucleic acid sequence of the binding site for a universal primer that is capable of hybridizing to a sequencing or PCR primer may be referred to as a primer binding site. A binding site for a universal primer may be at least about 1, 2, 3, 4, 5, 10, 15, 20, 25, 30, 35, 40, 45, 50 or more nucleotides in length. A binding site for a universal primer may comprise at least about 10 nucleotides. A binding site for a universal primer may be at most about 1, 2, 3, 4, 5, 10, 15, 20, 25, 30, 35, 40, 45, 50 or more nucleotides in length.

An oligonucleotide probe can comprise a dimension label. A dimension label can comprise a nucleic acid sequence that provides information about a dimension in which the stochastic labeling occurred. For example, a dimension label can provide information about the time at which a target was stochastically barcoded. A dimension label can be associated with a time of stochastic barcoding in a sample. A dimension label can activated at the time of molecular labeling. Different dimension labels can be activated at different times. The dimension label provides information about the order in which targets, groups of targets, and/or samples were stochastically barcoded. For example, a population of cells can be stochastically barcoded at the G0 phase of the cell cycle. The cells can be pulsed again with stochastic barcodes at the G1 phase of the cell cycle. The cells can be pulsed again with stochastic barcodes at the S phase of the cell cycle, and so on. Stochastic barcodes at each pulse (e.g., each phase of the cell cycle), can comprise different dimension labels. In this way, the dimension label provides information about which targets were labelled at which phase of the cell cycle. Dimension labels can interrogate many different biological times. Exemplary biological times can include, but are not limited to, the cell cycle, transcription (e.g., transcription initiation), and transcript degradation. In another example, a sample (e.g., a cell, a population of cells) can be stochastically labeled before and/or after treatment with a drug and/or therapy. The changes in the number of copies of distinct targets can be indicative of the sample's response to the drug and/or therapy.

A dimension label can be activatable. An activatable dimension label can be activated at a specific timepoint. The activatable dimension label may be constitutively activated (e.g., not turned off). The activatable dimension label can be reversibly activated (e.g., the activatable dimension label can be turned on and turned off). The dimension label can be reversibly activatable at least 1, 2, 3, 4, 5, 6, 7, 8, 9, or 10, or more times. The dimension label can be reversibly activatable at least 1, 2, 3, 4, 5, 6, 7, 8, 9, or 10 or more times. The dimension label can be activated with fluorescence, light, a chemical event (e.g., cleavage, ligation of another molecule, addition of modifications (e.g., pegylated, sumoylated, acetylated, methylated, deacetylated, demethylated), a photochemical event (e.g., photocaging, photocleavage), and introduction of a non-natural nucleotide.

The dimension label can be identical for all oligonucleotide probes in a plurality of oligonucleotide probes, but different for oligonucleotide probes in a different plurality of oligonucleotide probes. In some embodiments, at least 60%, 70%, 80%, 85%, 90%, 95%, 97%, 99%, or 100% of oligonucleotide probes in a plurality of oligonucleotide probes may comprise the same dimension label. In some embodiments, at least 60% of oligonucleotide probes in a plurality of oligonucleotide probes may comprise the same dimension label. In some embodiments, at least 95% of oligonucleotide probes in a plurality of oligonucleotide probes may comprise the same dimension label.

A dimension label may be at least about 1, 2, 3, 4, 5, 10, 15, 20, 25, 30, 35, 40, 45, 50 or more nucleotides in length. A dimension label may be at most about 300, 200, 100, 90, 80, 70, 60, 50, 40, 30, 20, 15, 12, 10, 9, 8, 7, 6, 5, 4 or fewer or more nucleotides in length. A dimension label may comprise from about 5 to about 200 nucleotides. A dimension label may comprise from about 10 to about 150 nucleotides. A dimension label may comprise from about 20 to about 125 nucleotides in length.

An oligonucleotide probe can comprise a spatial label. A spatial label can comprise a nucleic acid sequence that provides information about the spatial orientation of a target molecule which is associated with the molecular label sequence. A spatial label can be associated with a coordinate in a sample. The coordinate can be a fixed coordinate. For example a coordinate can be fixed in reference to a substrate. A spatial label can be in reference to a two or three-dimensional grid. A coordinate can be fixed in reference to a landmark. The landmark can be identifiable in space. A landmark can be a structure which can be imaged. A landmark can be a biological structure, for example an anatomical landmark. A landmark can be a cellular landmark, for instance an organelle. A landmark can be a non-natural landmark such as a structure with an identifiable identifier such as a color code, bar code, magnetic property, fluorescents, radioactivity, or a unique size or shape. A spatial label can be associated with a physical partition (e.g. a well, a container, or a droplet). In some instances, multiple spatial labels are used together to encode one or more positions in space.

The spatial label can be identical for all oligonucleotide probes in a plurality of oligonucleotide probes, but different for oligonucleotide probes in a different plurality of oligonucleotide probes. In some embodiments, at least 60%, 70%, 80%, 85%, 90%, 95%, 97%, 99% or 100% of oligonucleotide probes in a plurality of oligonucleotide probes may comprise the same spatial label. In some embodiments, at least 60% of oligonucleotide probes in a plurality of oligonucleotide probes may comprise the same spatial label. In some embodiments, at least 95% of oligonucleotide probes in a plurality of oligonucleotide probes may comprise the same spatial label.

A spatial label may be at least about 1, 2, 3, 4, 5, 10, 15, 20, 25, 30, 35, 40, 45, 50 or more nucleotides in length. A spatial label may be at most about 300, 200, 100, 90, 80, 70, 60, 50, 40, 30, 20, 15, 12, 10, 9, 8, 7, 6, 5, 4 or fewer or more nucleotides in length. A spatial label may comprise from about 5 to about 200 nucleotides. A spatial label may comprise from about 10 to about 150 nucleotides. A spatial label may comprise from about 20 to about 125 nucleotides in length.

An oligonucleotide probe may comprise a cellular label. A cellular label may comprise a nucleic acid sequence that provides information for determining which target nucleic acid originated from which cell. In some embodiments, the cellular label is identical for all oligonucleotide probes in a plurality of oligonucleotide probes, but different for oligonucleotide probes in a different plurality of oligonucleotide probes. In some embodiments, at least 60%, 70%, 80%, 85%, 90%, 95%, 97%, 99% or 100% of oligonucleotide probes in a plurality of oligonucleotide probes may comprise the same cellular label. In some embodiments, at least 60% of oligonucleotide probes in a plurality of oligonucleotide probes may comprise the same cellular label. In some embodiment, at least 95% of oligonucleotide probes in a plurality of oligonucleotide probes may comprise the same cellular label.

A cellular label may be at least about 1, 2, 3, 4, 5, 10, 15, 20, 25, 30, 35, 40, 45, 50 or more nucleotides in length. A cellular label may be at most about 300, 200, 100, 90, 80, 70, 60, 50, 40, 30, 20, 15, 12, 10, 9, 8, 7, 6, 5, 4 or fewer or more nucleotides in length. A cellular label may comprise from about 5 to about 200 nucleotides. A cellular label may comprise from about 10 to about 150 nucleotides. A cellular label may comprise from about 20 to about 125 nucleotides in length.

When an oligonucleotide probe comprises more than one of a type of label (e.g., more than one cellular label or more than one molecular label), the labels may be interspersed with a linker label sequence. A linker label sequence may be at least about 5, 10, 15, 20, 25, 30, 35, 40, 45, 50 or more nucleotides in length. A linker label sequence may be at most about 5, 10, 15, 20, 25, 30, 35, 40, 45, 50 or more nucleotides in length. In some instances, a linker label sequence is 12 nucleotides in length. A linker label sequence may be used to facilitate the synthesis of the molecular barcode. The linker label can comprise an error-correcting (e.g., Hamming) code.

### Sequencing

The amplicons comprising the molecular label sequence associated with the target nucleic acid can be subject to sequencing reactions to determine the target nucleic acid sequence or part thereof, the molecular label sequence or part thereof, or both. Any suitable sequencing method known in the art can be used, preferably high-throughput approaches. For example, cyclic array sequencing using platforms such as Roche 454, Illumina Solexa, ABI-SOLiD, ION Torrent, Complete Genomics, Pacific Bioscience, Helicos, or the Polonator platform, may also be utilized. Sequencing may comprise MiSeq sequencing. Sequencing may comprise HiSeq sequencing.

In some embodiments, sequencing can comprise sequencing at least about 10, 20, 30, 40, 50, 60, 70, 80, 90, 100, or more nucleotides or base pairs of the labeled nucleic acid and/or molecular label sequence. In some embodiments, sequencing can comprise sequencing at most about 10, 20, 30, 40, 50, 60, 70, 80, 90, 100, or more nucleotides or base pairs of the labeled nucleic acid and/or molecular label sequence. In some embodiments, sequencing can comprise sequencing at least about 200, 300, 400, 500, 600, 700, 800, 900, 1,000 or more nucleotides or base pairs of the labeled nucleic acid and/or molecular label sequence. In some embodiments, sequencing can comprise sequencing at most about 200, 300, 400, 500, 600, 700, 800, 900, 1,000 or more nucleotides or base pairs of the labeled nucleic acid and/or stochastic label sequence.

In some embodiments, sequencing can comprise at least about 200, 300, 400, 500, 600, 700, 800, 900, 1,000 or more sequencing reads per run. In some embodiments, sequencing can comprise at most about 200, 300, 400, 500, 600, 700, 800, 900, 1,000 or more sequencing reads per run. In some embodiments, sequencing comprises sequencing at least about 1,500; 2,000; 3,000; 4,000; 5,000; 6,000; 7,000; 8,000; 9,000; or 10,000 or more sequencing reads per run. In some embodiments, sequencing comprises sequencing at most about 1,500; 2,000; 3,000; 4,000; 5,000; 6,000; 7,000; 8,000; 9,000; or 10,000 or more sequencing reads per run. In some embodiments, sequencing can comprise sequencing at least 10, 50, 100, 150, 200, 250, 300, 350, 400, 450, 500, 550, 600, 650, 700, 750, 800, 850, 900, 950 or 1000 or more millions of sequencing reads per run. In some embodiments, sequencing can comprise sequencing at most 10, 50, 100, 150, 200, 250, 300, 350, 400, 450, 500, 550, 600, 650, 700, 750, 800, 850, 900, 950 or 1000 or more millions of sequencing reads per run. In some embodiments, sequencing can comprise sequencing at least 100, 200, 300, 400, 500, 600, 700, 800, 900, 1000, 1100, 1200, 1300, 1400, 1500, 1600, 2000, 3000, 4000, or 5000 or more millions of sequencing reads in total. In some embodiments, sequencing can comprise sequencing at most 100, 200, 300, 400, 500, 600, 700, 800, 900, 1000, 1100, 1200, 1300, 1400, 1500, 1600, 2000, 3000, 4000, or 5000 or more millions of sequencing reads in total. In some embodiments, sequencing can comprise less than or equal to about 1,600,000,000 sequencing reads per run. In some embodiments, sequencing can comprise less than or equal to about 200,000,000 reads per run.

### Kits

Some embodiments disclosed herein provide kits for quantitative analysis of a plurality of nucleic acid target molecules in a sample comprising a plurality of oligonucleotide probes, wherein each of the plurality of oligonucleotide probes comprises a target specific region, a molecular label sequence, a binding site for a first universal primer, and a binding site for a second universal primer, wherein the molecular label sequence is selected from a diverse set of unique molecular label sequences. The molecular label sequences of two of the plurality of oligonucleotide probes can be different.

In some embodiments, the kits further comprise a plurality of capture probes each comprising a second target specific region. The second target specific region can bind to the same target nucleic acid molecules that the oligonucleotide probes bind to. In some embodiments, the second target specific region can bind to all target nucleic acid molecules in a sample. For example, the second target specific region can comprise an oligo dT which can hybridize with mRNAs comprising poly-adenylated ends. The second target specific region can be gene-specific. For example, the second target specific region can be configured to hybridize to a specific region of a target. The second target specific region can be at least 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 or more nucleotides in length. The second target specific region can be at most 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 or more nucleotides in length. The second target specific region can be from 5-30 nucleotides in length.

The oligonucleotide probes disclosed herein can comprise a molecular label sequence, a target specific region, and a binding site for a universal primer. Without being bound by theory, the plurality of oligonucleotides comprises a unique set of molecular label sequence-target specific region combination. For example, each of the plurality of oligonucleotide probes may comprise a different molecular label sequence-target specific region combination. In some embodiments, the kits comprise at least 1,000 oligonucleotide probes. In some embodiments, the kits comprise at least 10,000 oligonucleotide probes. In some embodiments, the kits comprise at least 100,000 oligonucleotide probes. In some embodiments, the kits comprise at least 1,000,000 oligonucleotide probes. The oligonucleotide probes can have a variety of lengths. For example, an oligonucleotide probe can have a length that is, is about, is less than, is more than, 30 nt, 40 nt, 50 nt, 60 nt, 70 nt, 80 nt, 90 nt, 100 nt, 200 nt, 300 nt, 400 nt, 500 nt, 600 nt, 700 nt, 800 nt, 900 nt, 1,000 nt, or a range between any two of the above values

### Samples

A sample for use in the method, compositions, and kits of the disclosure can comprise one or more cells. In some embodiments, the nucleic acid target molecules are from an environmental sample, a plant, a non-human animal, a bacterium, archaea, a fungus, or a virus. The sample may be a purified sample or a crude sample containing lysate, for example derived from a buccal swap, paper, fabric or other substrate that may be impregnated with saliva, blood, or other bodily fluids. In some embodiments, the sample may be a formalin-fixed paraffin-embedded (FFPE) sample. As such, in some embodiments, the sample may comprise low amounts of, or fragmented portions of nucleic acid targets, such as genomic DNA or mRNA. For example, the sample may comprise an amount of nucleic acid (e.g., mRNA or genomic DNA) that is, is about, or is less than, 1 pg, 2 pg, 3 pg, 4 pg, 5 pg, 6 pg, 7 pg, 8 pg, 9 pg, 10 pg, 11 pg, 12 pg, 13 pg, 14 pg, 15 pg, 16 pg, 17 pg, 18 pg, 19 pg, 20 pg, 30 pg, 40 pg, 50 pg, 60 pg, 70 pg, 80 pg, 90 pg, 100 pg, 200 pg, 300 pg, 400 pg, 500 pg, 600 pg, 700 pg, 800 pg, 900 pg, 1 ng, 10 ng, 100 ng, or is in a range defined by any two of these values, for example, 10 pg to 100 pg, 10 pg to 1 ng, 100 pg to 1 ng, 1 ng to 10 ng, 10 ng to 100 ng, etc. In some embodiments, the sample is a forensic sample.

In some embodiments, the cells are cancer cells excised from a cancerous tissue, for example, breast cancer, lung cancer, colon cancer, prostate cancer, ovarian cancer, pancreatic cancer, brain cancer, melanoma and non-melanoma skin cancers, and the like. In some instances, the cells are derived from a cancer but collected from a bodily fluid (e.g. circulating tumor cells). Non-limiting examples of cancers can include, adenoma, adenocarcinoma, squamous cell carcinoma, basal cell carcinoma, small cell carcinoma, large cell undifferentiated carcinoma, chondrosarcoma, and fibrosarcoma. In some embodiments, the cells are cells that have been infected with virus and contain viral oligonucleotides. In some embodiments, the viral infection can be caused by a virus selected from the group consisting of double-stranded DNA viruses (e.g. adenoviruses, herpes viruses, pox viruses), single-stranded (+ strand or “sense”) DNA viruses (e.g. parvoviruses), double-stranded RNA viruses (e.g. reoviruses), single-stranded (+ strand or sense) RNA viruses (e.g. picornaviruses, togaviruses), single-stranded (− strand or antisense) RNA viruses (e.g. orthomyxoviruses, rhabdoviruses), single-stranded ((+ strand or sense) RNA viruses with a DNA intermediate in their life-cycle) RNA-RT viruses (e.g. retroviruses), and double-stranded DNA-RT viruses (e.g. hepadnaviruses). Exemplary viruses can include, but are not limited to, SARS, HIV, coronaviruses, Ebola, Malaria, Dengue, Hepatitis C, Hepatitis B, and Influenza.

In some embodiments, the cells are bacterial cells. These can include cells from gram-positive bacterial and/or gram-negative bacteria. Examples of bacteria that may be analyzed using the disclosed methods, devices, and systems include, but are not limited to, Actinomedurae, Actinomyces israelii, Bacillus anthracis, Bacillus cereus, Clostridium botulinum, Clostridium difficile, Clostridium perfringens, Clostridium tetani, Corynebacterium, Enterococcus faecalis, Listeria monocytogenes, Nocardia, Propionibacterium acnes, Staphylococcus aureus, Staphylococcus epiderm, Streptococcus mutans, Streptococcus pneumoniae and the like. Gram negative bacteria include, but are not limited to, Afipia felis, Bacteroides, Bartonella bacilliformis, Bortadella pertussis, Borrelia burgdorferi, Borrelia recurrentis, Brucella, Calymmatobacterium granulomatis, Campylobacter, Escherichia coli, Francisella tularensis, Gardnerella vaginalis, Haemophilius aegyptius, Haemophilius ducreyi, Haemophilius influenziae, Heliobacter pylori, Legionella pneumophila, Leptospira interrogans, Neisseria meningitidia, Porphyromonas gingivalis, Providencia sturti, Pseudomonas aeruginosa, Salmonella enteridis, Salmonella typhi, Serratia marcescens, Shigella boydii, Streptobacillus moniliformis, Streptococcus pyogenes, Treponema pallidum, Vibrio cholerae, Yersinia enterocolitica, Yersinia pestis and the like. Other bacteria may include Myobacterium avium, Myobacterium leprae, Myobacterium tuberculosis, Bartonella henseiae, Chlamydia psittaci, Chlamydia trachomatis, Coxiella burnetii, Mycoplasma pneumoniae, Rickettsia akari, Rickettsia prowazekii, Rickettsia rickettsii, Rickettsia tsutsugamushi, Rickettsia typhi, Ureaplasma urealyticum, Diplococcus pneumoniae, Ehrlichia chafensis, Enterococcus faecium, Meningococci and the like.

In some embodiments, the cells are cells from fungi. Non-limiting examples of fungi that may be analyzed using the disclosed methods, devices, and systems include, but are not limited to, Aspergilli, Candidae, Candida albicans, Coccidioides immitis, Cryptococci, and combinations thereof.

In some embodiments, the cells are cells from protozoans or other parasites. Examples of parasites to be analyzed using the methods, devices, and systems of the present disclosure include, but are not limited to, Balantidium coli, Cryptosporidium parvum, Cyclospora cayatanensis, Encephalitozoa, Entamoeba histolytica, Enterocytozoon bieneusi, Giardia lamblia, Leishmaniae, Plasmodii, Toxoplasma gondii, Trypanosomae, trapezoidal amoeba, worms (e.g., helminthes), particularly parasitic worms including, but not limited to, Nematoda (roundworms, e.g., whipworms, hookworms, pinworms, ascarids, filarids and the like), Cestoda (e.g., tapeworms).

As used herein, the term “cell” can refer to one or more cells. In some embodiments, the cells are normal cells, for example, human cells in different stages of development, or human cells from different organs or tissue types (e.g. white blood cells, red blood cells, platelets, epithelial cells, endothelial cells, neurons, glial cells, fibroblasts, skeletal muscle cells, smooth muscle cells, gametes, or cells from the heart, lungs, brain, liver, kidney, spleen, pancreas, thymus, bladder, stomach, colon, small intestine). In some embodiments, the cells can be undifferentiated human stem cells, or human stem cells that have been induced to differentiate. In some embodiments, the cells can be fetal human cells. The fetal human cells can be obtained from a mother pregnant with the fetus. In some embodiments, the cells are rare cells. A rare cell can be, for example, a circulating tumor cell (CTC), circulating epithelial cell, circulating endothelial cell, circulating endometrial cell, circulating stem cell, stem cell, undifferentiated stem cell, cancer stem cell, bone marrow cell, progenitor cell, foam cell, mesenchymal cell, trophoblast, immune system cell (host or graft), cellular fragment, cellular organelle (e.g. mitochondria or nuclei), pathogen infected cell, and the like.

In some embodiments, the cells are non-human cells, for example, other types of mammalian cells (e.g. mouse, rat, pig, dog, cow, or horse). In some embodiments, the cells are other types of animal or plant cells. In some embodiments, the cells can be any prokaryotic or eukaryotic cells.

In some embodiments, a first cell sample is obtained from a person not having a disease or condition, and a second cell sample is obtained from a person having the disease or condition. In some embodiments, the persons are different. In some embodiments, the persons are the same but cell samples are taken at different time points. In some embodiments, the persons are patients, and the cell samples are patient samples. The disease or condition can be a cancer, a bacterial infection, a viral infection, an inflammatory disease, a neurodegenerative disease, a fungal disease, a parasitic disease, a genetic disorder, or any combination thereof.

In some embodiments, cells suitable for use in the presently disclosed methods can range in size, for example ranging from about 2 micrometers to about 100 micrometers in diameter. In some embodiments, the cells can have diameters of at least 2 micrometers, at least 5 micrometers, at least 10 micrometers, at least 15 micrometers, at least 20 micrometers, at least 30 micrometers, at least 40 micrometers, at least 50 micrometers, at least 60 micrometers, at least 70 micrometers, at least 80 micrometers, at least 90 micrometers, or at least 100 micrometers. In some embodiments, the cells can have diameters of at most 100 micrometers, at most 90 micrometers, at most 80 micrometers, at most 70 micrometers, at most 60 micrometers, at most 50 micrometers, at most 40 micrometers, at most 30 micrometers, at most 20 micrometers, at most 15 micrometers, at most 10 micrometers, at most 5 micrometers, or at most 2 micrometers. The cells can have a diameter of any value within a range, for example from about 5 micrometers to about 85 micrometers. In some embodiments, the cells have diameters of about 10 micrometers.

In some embodiments, the cells are sorted prior to associating one or more of the cells with a bead and/or in a microwell. For example the cells can be sorted by fluorescence-activated cell sorting or magnetic-activated cell sorting, or e.g., by flow cytometry. The cells can be filtered by size. In some instances a retentate contains the cells to be associated with the bead. In some instances the flow through contains the cells to be associated with the bead.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method of quantitative analysis of a plurality of nucleic acid target molecules in a sample comprising:
providing a sample comprising a plurality of nucleic acid target molecules;
providing a plurality of oligonucleotide probes, wherein each of the plurality of oligonucleotide probes comprises a target specific region, a molecular label sequence, and a binding site for a first universal primer, wherein the molecular label sequence is selected from a diverse set of unique molecular label sequences, and wherein the molecular label sequences of two of the plurality of oligonucleotide probes are different;
contacting the plurality of oligonucleotide probes with the plurality of nucleic acid target molecules for hybridization;
removing oligonucleotide probes that are not hybridized to the plurality of nucleic acid target molecules;
amplifying oligonucleotide probes that are hybridized to the plurality of nucleic acid target molecules using the first universal primer to generate a plurality of amplicons, wherein each of the plurality of amplicons comprises a target specific region and a molecular label sequence; and
determining the number of unique molecular label sequences for each target specific region,
whereby the quantity of each nucleic acid target molecule in the sample is determined.

2. The method of claim 1, further comprising immobilizing the plurality of nucleic acid target molecules on a solid support via an affinity moiety and a binding partner of the affinity moiety.

3. The method of claim 2, wherein the plurality of nucleic acid target molecules comprises the affinity moiety.

4. The method of claim 2 or 3, wherein the affinity moiety comprises a functional group of biotin, streptavidin, heparin, an aptamer, a click-chemistry moiety, digoxigenin, primary amine, carboxyl, hydroxyl, aldehyde, ketone, or any combination thereof.

5. The method of claim 4, wherein the plurality of nucleic acid target molecules is biotinylated.

6. The method of claim 4, wherein the plurality of nucleic acid target molecules is hybridized to a plurality of biotinylated capture probes.

7. The method of claim 6, wherein each of the plurality of biotinylated capture probes comprises a second target specific region.

8. The method of claim 7, wherein the second target specific region comprises poly dT.

9. The method of any one of claims 3-8, wherein the solid support comprises the binding partner of the affinity moiety.

10. The method of any one of claims 2-9, wherein removing oligonucleotide probes that are not hybridized to the plurality of nucleic acid target molecules comprises washing the solid support.

11. The method of any one of claims 1-10, wherein each of the plurality of oligonucleotide probes comprises a binding site for a second universal primer.

12. The method of claim 11, wherein the amplifying comprises PCR amplification of at least a portion of the oligonucleotide probes that are hybridized to the plurality of nucleic acid target molecules using the first universal primer and the second universal primer.

13. The method of any one of claims 1-12, wherein each of the plurality of oligonucleotide probes comprises a cellular label, a sample label, a location label, or any combination thereof.

14. The method of any one of claims 1-13, wherein the target specific region comprises 20 nt to 500 nt.

15. The method of any one of claims 1-14, wherein the sample comprises a single cell, a plurality of cells, a tissue sample, or any combination thereof.

16. The method of any one of claims 1-15, wherein the diverse set of unique molecular label sequences comprises at least 100 unique molecular label sequences.

17. The method of any one of claims 1-15, wherein the diverse set of unique molecular label sequences comprises at least 1,000 unique molecular label sequences.

18. The method of any one of claims 1-15, wherein the diverse set of unique molecular label sequences comprises at least 10,000 unique molecular label sequences.

19. The method of any one of claims 1-18, wherein at least 10 of the plurality of oligonucleotide probes comprise different target specific regions.

20. The method of any one of claims 1-18, wherein at least 100 of the plurality of oligonucleotide probes comprise different target specific regions.

21. The method of any one of claims 1-18, wherein at least 1,000 of the plurality of oligonucleotide probes comprise different target specific regions.

22. The method of any one of claims 1-21, wherein each of the plurality of RNA target molecules hybridizes to a single target specific region.

23. The method of any one of claims 1-21, wherein each of the plurality of RNA target molecules hybridizes to more than one different target specific regions.

24. The method of any one of claim 1-23, further comprising sequencing the plurality of amplicons.

25. The method of claim 24, wherein the sequencing comprises sequencing at least a portion of the molecular label sequence and at least a portion of the target specific region.

26. The method of claim 24 or 25, further comprising associating the sequence of the molecular label sequence with the sequence of the target specific region.

27. A kit for quantitative analysis of a plurality of nucleic acid target molecules in a sample comprising a plurality of oligonucleotide probes, wherein each of the plurality of oligonucleotide probes comprises a target specific region, a molecular label sequence, a binding site for a first universal primer, and a binding site for a second universal primer, wherein the molecular label sequence is selected from a diverse set of unique molecular label sequences, and wherein the molecular label sequences of two of the plurality of oligonucleotide probes are different.

28. The kit of claim 27, further comprising a plurality of capture probes each comprising a second target specific region.

29. The kit of claim 28, wherein the plurality of capture probes is biotinylated.

30. The kit of claim 28 or 29, wherein the second target specific region comprises poly dT.

31. The kit of any one of claims 27-30, wherein each of the plurality of oligonucleotide probes comprises a cellular label, a sample label, a location label, or any combination thereof.

32. The kit of any one of claims 27-31, wherein the target specific region comprises 20 nt to 500 nt.

33. The kit of any one of claims 27-31, wherein the diverse set of unique molecular label sequences comprises at least 100 unique molecular label sequences.

34. The kit of any one of claims 27-31, wherein the diverse set of unique molecular label sequences comprises at least 1,000 unique molecular label sequences.

35. The kit of any one of claims 27-31, wherein the diverse set of unique molecular label sequences comprises at least 10,000 unique molecular label sequences.

36. The kit of any one of claims 27-35, wherein at least 10 of the plurality of oligonucleotide probes comprise different target specific regions.

37. The kit of any one of claims 27-35, wherein at least 100 of the plurality of oligonucleotide probes comprise different target specific regions.

38. The kit of any one of claims 27-35, wherein at least 1,000 of the plurality of oligonucleotide probes comprise different target specific regions.

39. The kit of any one of claims 27-38, wherein each of the plurality of oligonucleotide probes comprises a different molecular label sequence-target specific region combination.

40. The kit of any one of claims 27-39, comprising at least 1,000 oligonucleotide probes.

41. The kit of any one of claims 27-39, comprising at least 10,000 oligonucleotide probes.

42. The kit of any one of claims 27-39, comprising at least 100,000 oligonucleotide probes.

43. The kit of any one of claims 27-39, comprising at least 1,000,000 oligonucleotide probes.
